(19) United States
(12) Patent Application Publication
(10) Pub. No.: US 2006/0155336 A1
(43) Pub. Date: Jul. 13, 2006

(54) Medical resuscitation system and patient information module

(76) Inventor: Roger Lee Heath, Tempe, AZ (US)

Correspondence Address: ADAM K. SACHAROFF;MUCH SHELIST FREED DENENBERG AMENT&RUBENSTEIN,PC, 191 N. WACKER DRIVE, SUITE 1800, CHICAGO, IL, 60606-1615, US

(21) Appl. No.: 11/331,737

(22) Filed: Jan. 13, 2006

Related U.S. Application Data
(60) Provisional application No. 60/644,122, filed on Jan. 13, 2005.

(51) Int. Cl. A61N 1/39

(52) U.S. Cl. 607/5

(57) ABSTRACT

A medical resuscitation system includes an external physiological stimulator system module for resuscitating a patient and an external communication system module. The external physiological stimulator and communication system modules are removeably coupled together. The external communication system module can flow a signal between it and an electronic patient information module carried by a patient. The external physiological stimulator system module can resuscitate the patient in response to this signal.

### CROSS-REFERENCE TO RELATED APPLICATIONS

This application claims the benefit of U.S. Provisional Application, Ser. No. 60/644,122, filed on Jan. 13, 2005.

### BACKGROUND OF THE INVENTION

1. Field of the Invention

This invention relates generally to surgery and, more particularly, to a medical resuscitation system which responds to an electronic patient information module.

2. Description of the Related Art

A number of patients suffer from arrhythmias, such as ventricular fibrillation (VF) and atrial fibrillation (AF), each year and are often referred to as cardiac patients. It is known that the chances of survival increase if the time between the onset of VF and medical treatment decreases. For example, a cardiac patient's chances of survival decrease about 10% for every minute that elapses after VF begins and before defibrillation is initiated. Since most cardiac patients are away from a hospital at the onset of VF, automatic external defibrillators (AEDs) have been developed which can be brought to the patient. However, there are several problems not addressed by current AEDs.

For example, in a course of treatment, it is often desirable to treat the patient according to a protocol, which is a plan for a course of medical treatment. In the United States, the protocols are generally defined by the American Heart Association (AHA). One resource for these protocols is titled “2005 American Heart Association Guidelines for Cardiopulmonary Resuscitation and Emergency Cardiovascular Care”, which is currently updated yearly. In a typical protocol for a cardiac patient, some of the steps include calling for help and providing cardiopulmonary resuscitation (CPR). The protocol also typically includes steps of providing defibrillation, ventilation, and patient monitoring.

Current AEDs, however, can only perform the defibrillation step and some monitoring. Further, current AEDs are lacking in their ability to communicate information about the patient to a remote location, although there are several monitors which do so. For example, there are several providers of patient monitoring services. Such providers include Lifeline Systems, Inc. and the Medicalert Foundation.

In February of 2002, CardioNet, Inc. received approval from the Federal Drug Administration to market its CardioNet Ambulatory Monitor with Arrhythmia Detection. This monitor is useful for patients who have demonstrated a need for cardiac monitoring and have a low risk of developing primary ventricular fibrillation or sustained ventricular tachycardia. It is also useful for patients who need monitoring for non life-threatening arrhythmias, such as atrial fibrillation, other supra-ventricular arrhythmias, and the evaluation of various bradyarrhythmias.

However, there are several problems that the CardioNet System does not address. For example, it is contraindicated for use with patients who are highly likely to experience ventricular tachycardia or fibrillation. Accordingly, it is highly desirable to have a medical resuscitation system that can provide patient monitoring and implement more steps in a protocol to treat the patient.

### BRIEF SUMMARY OF THE INVENTION

The present invention provides a medical resuscitation system having a communication system. The communication system can communicate with an electronic patient information module carried by a patient. The electronic patient information module has medical information corresponding to the patient which it provides to the medical resuscitation system. This information enables the patient to be treated faster and more effectively.

The treatment is faster because the information is provided to medical personnel assisting the patient. In some examples, the medical personnel can be at a remote location or they can be on the scene. If the medical personnel are at the remote location, the information is sent to them through a communication system. The remote medical personnel can then send a signal to the medical resuscitation system to implement a desired protocol to treat the patient. If the medical personnel on the scene, then the information can be displayed on a display included in the resuscitation system. The treatment is more effective because the medical personnel have the patient's medical information, as provided by the electronic patient information module, and can choose an appropriate protocol based on it.

These and other features, aspects, and advantages of the present invention will become better understood with reference to the following drawings, description, and claims.

### BRIEF DESCRIPTION OF THE DRAWINGS

FIG. 1 is a perspective view of a medical resuscitation system in communication with an electronic patient information module and remote communication system, in accordance with the present invention;

FIGS. 2a and 2b are block diagrams of the medical resuscitation system of FIG. 1 in communication with the remote communication system and electronic patient information module, in accordance with the present invention;

FIG. 3 is a more detailed view of an electrode system, in accordance with the present invention, for use with the medical resuscitation system of FIG. 1;

FIGS. 4a and 4b are more detailed views of a breathing circuit, in accordance with the present invention, for use with the medical system of FIG. 1;

FIGS. 5 and 6 are partial side views of the patient coupled to the medical resuscitation system of FIG. 1 with the electrode system of FIG. 3;

FIG. 7 is a side view of the patient of FIG. 1 with an array of implanted medical devices included therein;

FIG. 8a is a flowchart of a method of communicating with an electronic patient information module, in accordance with the present invention;

FIG. 8b is a flowchart of a method of resuscitating a patient, in accordance with the present invention;

FIG. 9a is a flowchart of various methods of communicating using a medical resuscitation system, in accordance with the present invention; and

FIG. 9b is a flowchart of various methods of helping a patient, in accordance with the present invention.

### DETAILED DESCRIPTION OF THE INVENTION

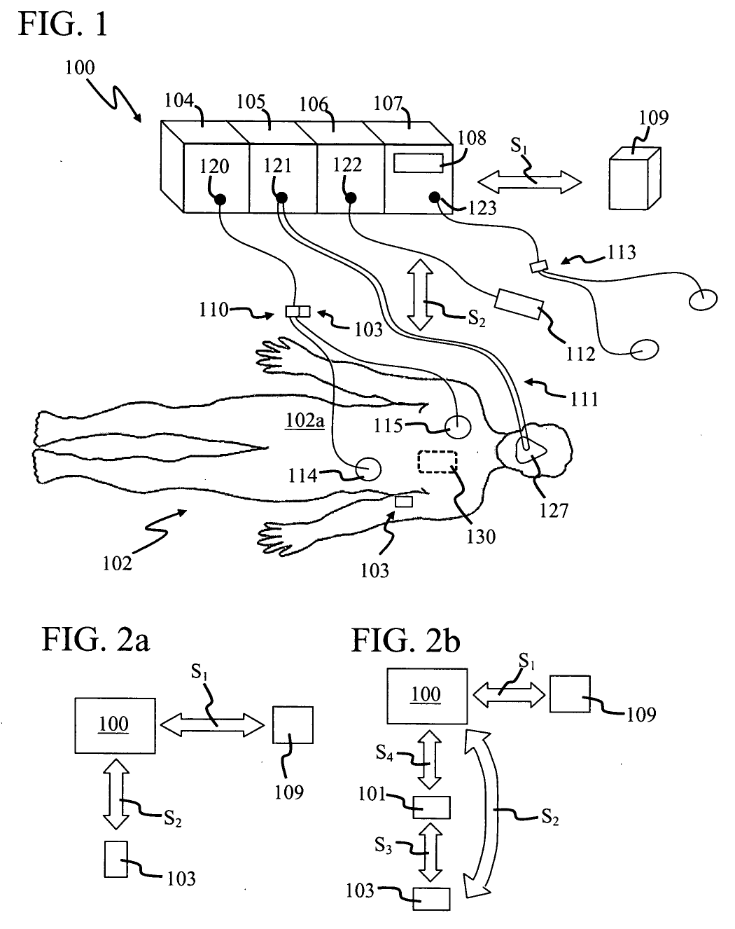

FIG. 1 is a perspective view of a medical resuscitation system 100, in accordance with the present invention. System 100 provides many different advantages which are discussed in more detail in U.S. patent application Ser. No. 11/235,005 (“the '005 application”) filed on Sep. 26, 2005 by the same inventor, and incorporated herein by reference. System 100 can resuscitate a patient 102 because it can ventilate, defibrillate, and/or pace him or her. In this example, system 100 includes a physiological stimulator 104, ventilator 105, communication system 106, and monitor 107. An electrode system 110 is coupled between stimulator 104 at a port 120 and patient 102 to flow a defibrillation and/or pacing signal therebetween. Further, a breathing circuit 111 is coupled between ventilator 105 at a port 121 and patient 102 to provide ventilation thereto. An electrode system 113 is coupled to monitor 107 at a port 123 and is used to monitor the vital signs of patient 102.

In one embodiment, system 100 is modular so stimulator 104, ventilator 105, communication system 106, and monitor 107 can be repeatably moved between engaged and disengaged positions relative to each other. System 100 is also portable so it can be moved from one location to another and brought to patient 102 to decrease the patient response time. The patient response time is further decreased because communication system 106 provides patient location and/or medical information, which allows patient 102 to be assisted faster and more effectively. The provides patient location and/or medical information is generally provided after system 100 is positioned near patient 102, as will be discussed below.

The assistance is provided faster because system 100 communicates the location of patient 102 to a remote communication system 109, so that medical personnel, referred to as local medical personnel, arrive faster. The local medical personnel are generally paramedics, but they can also be a first responder, which can be a non-medically trained layperson, for example. System 109 is preferably monitored by remote medical personnel, who are preferably doctors, nurses, and other trained medical personnel. System 100 can provide the patient location in many different ways. For example, it can include a global positioning system (GPS) or another system which provides position information. One such system is disclosed in U.S. Pat. No. 5,959,529, which is incorporated herein by reference.

In accordance with the invention, patient 102 is assisted more effectively because system 100 communicates with an electronic patient information module 103 carried by patient 102. Module 103 can be carried by patient 102 in many different ways. For example, it can be carried by patient 102 internally and/or externally, as will be discussed in more detail below.

Module 103 provides medical information corresponding to patient 102 to system 100. In one example, this information is used by the local medical personnel to treat patient 102 locally using system 100. In another example, this information is communicated to remote communication system 109 where the remote medical personnel use it to treat patient 102 remotely using system 100. In some examples, the communication of the medical information is protected to protect patient privacy. This can be done in many different ways.

For example, the medical information can be encrypted or the communication can take place using a secure communication link. The secure communication link can be established in response to a “handshake” which is a procedure generally used in computer networking and provides authentication. Module 103 can also store identification information relating to system 100 so that it can be determined which equipment was used to communicate with it and the date and/or time this communication took place.

In one embodiment, electronic patient information module 103 is a Radio Frequency IDentification (RFID) system, which is carried externally by patient 102. Module 103 can be carried externally by patient 102 in many different ways. For example, it can be carried by a bracelet or garment worn by patient 102. It can also be attached to the skin of patient 102. Module 103 may also be positioned near patient 102, such as when patient 103 is in a home or residence. If module 103 is positioned near patient 102, it is preferably positioned so that it allows him or her to be assisted faster and more effectively.

In mass casualty situations, module 103 can be carried by electrode system 110, as shown in FIG. 1. This is useful in these situations because it is often difficult to keep track of a large number of patients. Since electrode system 110 is typically coupled to a particular patient and left in place throughout his or her treatment, module 103 can be carried by electrode system 110. In this way, it provides patient tracking and still allows patient 102 to be assisted faster and more effectively.

In some situations, module 103 is carried by pediatric electrode pads, such as those for use by children and infants. Module 103 can communicate to system 100 that patient 102 is a child or infant so that system 100 will implement the appropriate protocol. The appropriate protocol for children and infants generally involves providing smaller amplitude defibrillation signals compared to those provided to adults. In still other situations, system 100 is used to provide the defibrillation signal to an internal electrode paddle through a cable system coupled between the internal electrode paddle and port 120. Internal electrode paddles are those typically used during surgery and are connected to the heart instead of the skin of the patient. In these situations, module 103 can be carried by the cable system and provide a signal to system 100 so that the appropriate protocol is implemented by it.

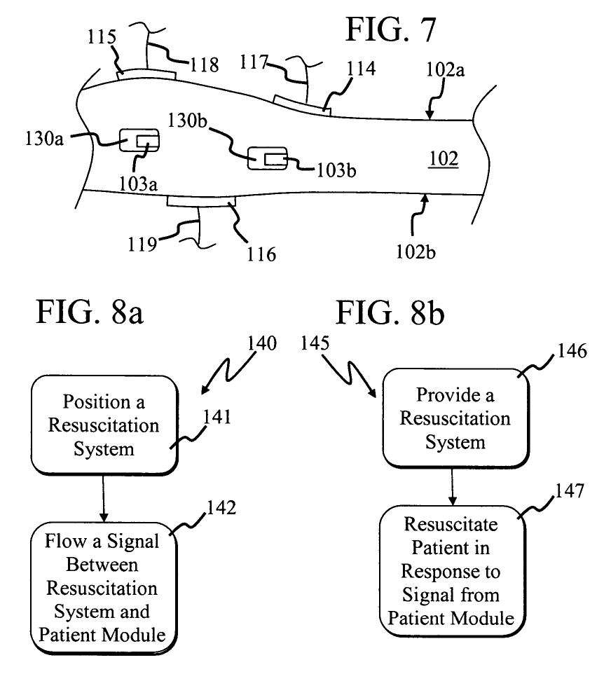

In other examples, module 103 is implanted into patient 102, generally under the skin. It is preferable that module 103 be implanted in the patient's upper inner arm as shown, but it can be implanted in other locations such as the patient's hand, chest, back, leg, etc. In still other examples, module 103 is integrated with an implanted medical device (IMD) 130, which is surgically inserted into patient 102 (FIGS. 5 and 7). This is useful so that it can be determined if patient 102 has IMD 130, although module 103 can provide information regarding the presence of IMD 130 if it is not integrated with it. This feature is useful because the presence or absence of IMD 130 often affects the choice of protocol used to treat patient 102, as will be discussed with FIG. 5.

In some situations, IMD 130 can be implanted in an emergency or, in others, it can be implanted prophylactically. Further, IMD 130 can store recommended treatment protocols which correspond to the particular patient. The recommended treatment protocols can include, for example, the type and/or amount of drugs which where useful in the past to treat this particular patient.

There are several different types of IMDs that can be implanted into patient 102, such as a pacemaker, defibrillator, and infusion pump, among others. Implanted pacemakers are described in more detail in U.S. Pat. Nos. 6,968,235, 6,922,592, 6,721,600, 6,675,049, 6,289,244, and 6,016,447 and implanted defibrillators are described in U.S. Pat. Nos. 5,817,132 and 5,174,288. Further, implanted infusion pumps are disclosed in U.S. Pat. Nos. 6,635,048 and 6,283,949. These patents are all incorporated herein by reference.

IMD 130 can also include one or more medical sensors, which determine the core physiological condition of patient 102 and provide this information to module 103. The core physiological condition can include the temperature, blood gas, blood pressure, glucose levels, etc. of patient 15. Examples of sensors include a blood glucose sensor, heart monitoring sensor, and breathing monitoring sensor, among others. A breathing monitoring sensor typically includes a transducer which senses sounds within patient 102 and provides this information to module 103 where it is then provided to system 100. The sounds can correspond to the heartbeat and/or breathing of patient 102, for example. Examples of physiological sensors are disclosed in U.S. Pat. Nos. 6,937,654, 6,953,455, 6,937,899, 6,964,641, 6,600,949, and 6,354,299, which are incorporated herein by reference.

In some embodiments, IMD 130 is activated and deactivated in response to a signal from module 103. For example, if IMD 130 includes an infusion pump, then it can be activated to provide insulin and then deactivated. In another example, IMD 130 includes a heart monitoring sensor that is activated to provide heart monitoring in response to a signal from module 130. In this way, IMD 130 can be carried by patient 102 in a deactivated mode and then activated by module 103 when needed. IMD 130 can then be deactivated when it is no longer needed. These steps can then be repeated.

Also, in most situations, it is necessary for local medical personnel to establish an intravenous (IV) line for chemical delivery into patient 102. However, this is often a burden because establishing an IV line is difficult and time consuming, especially in medical situations. In these situations, if IMD 130 includes an infusion pump, then it can be activated in response to a signal from module 103 to provide chemical delivery. Since this can be done faster then establishing an IV line, the chances of survival for patient 102 increase.

Module 103 provides many different types of information to system 100, such as identification, contact information, medical history, an event log, physiological data, presence or absence of an IMD, etc. Module 103 can also provide information from IMD 130 to system 100. The medical history can include the current and past medications that the patient is taking which can affect the choice of protocol used to treat patient 102. The event log typically includes what treatments have been implemented in treating patient 102 and is useful to provide to later medical personnel, such as those at a hospital.

The treatments can include the type and dose of medications, the time, date, and/or sequence of any resuscitation attempts, etc. Physiological data generally includes the core temperature, blood gas levels, blood oxygenation level, blood pressure, glucose levels, among others, corresponding to patient 102. Some or all of this data may be useful in the treatment of patient 102. In some examples, module 103 provides operational data regarding IMD 130 so it can be determined whether or not it is functioning and/or calibrated properly. In these ways, module 103 provides different types of information regarding patient 102 so he or she is treated more effectively. As will be discussed below, some or all of this information can be displayed by a display 108 included in system 100 so it is available to local medical personnel. Some or all of this information can also be provided to remote communication system 109.

There are several different RFID systems that can be used with electronic patient information module 103. These systems are generally used as an identification system which relies on storing and communicating information using an RFID chip, which is also referred to in the art as an RFID tag or transponder. A typical RFID system includes the RFID chip electrically coupled to an RFID antenna. The RFID chip generally includes electronic circuitry which operates as a transceiver and memory. The RFID antenna allows signals to flow between the RFID chip and another communication system, such as communication system 106.

RFID chips normally flow signals at a frequency of about 134.2 kHz, although other frequencies can be used, and generally have communication ranges from less than an inch to several feet or more depending on the amount of signal power. There are currently two types of RFID systems; one system is passive and does not use an internal power source and the other system is active and does use an internal power source. If module 103 is positioned outside of patient 102 (i.e. not implanted), then it can also include a scanable card.

One type of implantable RFID system is sold under the trademark VERICHIP is manufactured by Verichip Corporation. It should be noted that there are similar RFID systems made by other manufacturers which can be used. Other companies that manufacture RFID systems are Medtronic, Inc and Symbol Technologies, Inc. RFID Systems that can be used are disclosed in U.S. Pat. Nos. 6,922,592, 6,561,975, 6,450,953, 6,115,636, and 6,016,447, which are incorporated herein by reference.

In operation, a signal S₁ flows between resuscitation system 100 and remote communication system 109 and a signal S₂ flows between resuscitation system 100 and module 103, as shown in FIG. 1. Signal S₁ generally includes control, voice, and/or data information and signal S₂ generally includes control and/or data information. In one embodiment, external communication system 109 is an emergency services communication center, such as those commonly found at a hospital or medical call center. However, it should be noted that system 109 can be another type of communication center used by medical personnel. Communications systems 106 and 109 can communicate with each other in many different ways. In one example, they communicate with each other through a wireless link, although in other examples the communication can be through a land line, WiFi link, computer link, radio link, or combinations thereof.

Communication system 106 preferably allows system 100 to transmit and receive the different types of signals at the same time and at different times. In this embodiment, system 106 does this by providing multiple communication links which transmit and receive the control, data, and voice information. The multiple communication links can be provided in several different ways, such as by wireless network, a land line phone network, WiFi network, computer network, radio network, or combinations thereof.

WiFi allows data and voice to be transmitted over the same link without significant interference between the voice and data signals, so in some examples a single communication link can be used and the information is transmitted and received at the same time and at different times. A WiFi link can do this because it uses a known technology called voice over internet protocol (VOIP). Examples of communication systems similar to system 106 are described in U.S. Pat. Nos. 6,957,107, 6,564,104, 6,497,655, and 5,626,630, which are incorporated herein by reference.

It should be noted that it is preferred that communication system 106 provide multiple communication links for several reasons. One reason is communication redundancy in case one communication link is not available. A communication link may not be available for several different reasons, such as a hardware or software problem or failure, the remoteness of the location, weather, etc. Another reason is that different types of information, such as voice and data, can be transmitted and received at different times and at the same time. If the information is transmitted and received at the same time, then this speeds up the treatment of patient 102. For example, a remotely located person can communicate with patient 102 by voice while also receiving patient data from system 100 and/or sending control signals to system 100. The patient data can include the vital signs of patient 102, for example, and the control signals can include protocols to be implemented by system 100 to medically treat patient 102.

Systems 103 and 106 can also communicate with each other in many other different ways. For example, they can communicate directly through an RFID communicator or indirectly through a repeater system, as will be discussed in more detail below. In FIG. 1, system 100 includes an RFID communicator 112 coupled to communication system 106 at a port 122. RFID communicator 112 can operate in many different ways. For example, it can operate as an RFID reader for reading information stored by module 103. It can also operate as an RFID programmer for programming and storing information with module 103. This information can include patient data or control parameters for IMD 130. In this example, RFID communicator 112 is a hand-held device which communicates with module 103 wirelessly when positioned close enough to it. It should be noted that RFID communicator 112 is coupled directly to communication system 106 for illustrative purposes, but it could be coupled indirectly to system 106 through any of the other modules included in system 100.

In accordance with the invention, IMD communicator 112 is used with system 100 so that system 100 has the ability to control the operation of IMD 130. For example, if IMD 130 is a pacemaker, then system 100 can use IMD communicator 112 to control its operation. In another example, if IMD 130 is an infusion pump, then system 100 can use IMD communicator 112 to have the infusion pump provide patient 102 with a medicine, such as epinephrine, insulin, vasopressin, amiodarone, glucose, among others. These medications are typically administered as an inhalant through the air-way or intravenously to patients suffering from arrhythmia or other adverse medical conditions. In either case, the operation of IMD communicator 112 is controllable by the remote medical personnel monitoring communication system 109. In this way, they can provide patient 102 with the appropriate medication much faster so patient 102 does not have to wait for the arrival of the local medical personnel.

FIGS. 2a and 2b are block diagrams of different ways in which module 103 is in communication with resuscitation system 100. In FIG. 2a, module 103 is in communication with system 100 directly and in FIG. 2b, module 103 is in communication with system 100 indirectly through a repeater system 101. Repeater system 101 includes circuitry well-known in the art, such as an RFID transceiver, amplifier, and antenna, for flowing signals between it and communication system 100 and module 103. Repeater system 101 is useful in situations where the transmission range of module 103 is too small for signals transmitted therefrom to reach system 100 with enough signal power.

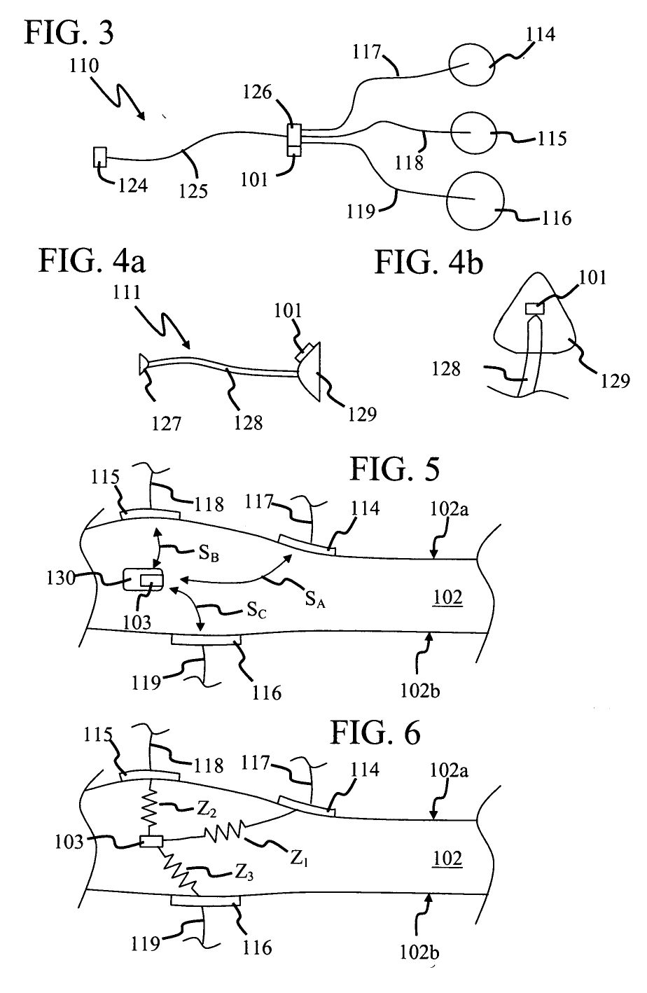

In one mode of operation, repeater system 101 receives a signal S₃ transmitted by module 103, amplifies it, and then transmits it to resuscitation system 100 as signal S₄. In another mode of operation, repeater system 101 receives signal S₄ transmitted by resuscitation system 100, amplifies it, and then transmits it to module 103 as signal S₃. In other modes of operation, signal S₂ can flow directly between systems 100 and 103 if the signal power is high enough. It should be noted that repeater system 101 can be positioned in many different locations, as will be discussed in more detail below with FIGS. 3, 4a, and 4b. Further, in other examples, an electrode system coupled between system 100 and patient 102 can be used to flow signal S₂ between systems 100 and 103, as will be discussed in more detail with FIGS. 5 and 6.

In FIG. 1, physiological stimulator 104 is in communication with the heart (not shown) of patient 102 through electrode system 110 coupled to anterior 102a of patient 102. Stimulator 104 provides many different circulatory resuscitation functions, such as defibrillation and/or pacing, for patient 102. Stimulator 104 can be of many different types, but is an automatic external defibrillator (AED) in this example. These types of defibrillators are made by many different manufacturers, such as Zoll Medical Corporation, Medtronic, Inc., and Philips Medical Systems, in Bothell, Wash. More information about AEDs is disclosed in U.S. Pat. Nos. 6,694,187, 6,668,192, 6,047,212, 5,782,878, 5,725,560, and 4,102,332, which are incorporated herein by reference.

FIG. 3 is a more detailed view of electrode system 110, in accordance with the present invention. In this embodiment, system 110 includes an electrode cable 125 with a cable connector 124 at one end and an electrode connector 126 at its opposed end. Cable connector 124 is dimensioned and shaped to be received by port 120 (FIG. 1) so it is moveable between engaged and disengaged positions relative to port 120. Port 120 is an output of stimulator 104 which outputs defibrillation and/or pacing signals therethrough and receives the return signal to complete the circuit.

An anterior apex electrode cable 117 is coupled to cable 125 through electrode connector 126 at one end and is attached to an anterior apex electrode pad 114 at its other end. An anterior sternum electrode cable 118 is coupled to cable 125 through electrode connector 126 at one end and is attached to an anterior sternum electrode pad 115 at its other end. In this way, electrode pads 114 and 115 are in communication with stimulator 104 when connector 124 is coupled to port 120. Further, electrode pads 114 and 115 are coupled to anterior 102a (FIGS. 5 and 6) of patient 102 so that they are in communication with the heart of patient 102, as shown in FIG. 1, and stimulator 104 can provide circulatory life support or resuscitation for patient 102. In some embodiments, electrode system 110 includes a posterior electrode cable 119 coupled to cable 125 through electrode connector 126 at one end and a porterior electrode pad 116 at its other end. Posterior electrode pad 116 is coupled to posterior torso region 102b (FIGS. 5 and 6).of patient 102. In this example, cable 125 has separate conductive lines (not shown) which flow the signals from cables 117, 118, and 119 separately. However, the separate conductive lines are shown as cable 125 for simplicity.

In this embodiment, repeater system 101 is carried by electrode system 110 and, in particular, system 101 is carried by connector 126. In other examples, however, it can be carried by electrode system 110 at other locations. For example, it can be carried by one of electrode pads 114, 115, and 116. Electrode system 110 can be of many different types made by the AED manufacturers mentioned above or others. More information about electrode systems can be found in U.S. Pat. Nos. 4,895,169, 4,852,585, 4,850,356, 4,834,103, 4,653,503, 4,494,552, and 4,419,998 by the inventor of the inventions included herein, each of which are incorporated herein by reference. U.S. Pat. No. 4,786,277 also discloses an electrode system and is incorporated herein by reference.

The present invention can also be used with the physiological stimulator electrode pads and medical system discussed in a copending patent application Ser. No. ______, entitled “Electrode System for a Physiological Stimulator”, filed on the same day as the present invention by the same inventor, and incorporated herein by reference.

As best seen in FIG. 1, ventilator 105 is in communication with patient 102 through breathing circuit 111. Ventilator 105 provides many different respiratory functions, such as breathing and oxygenation of the blood of patient 102, as needed. Ventilator 105 can be of many different types, but is an Airway Pressure Release Ventilation (APRV) type ventilator in this example. Further, breathing circuit 111 can be of many different types known in the art.

As best seen in FIGS. 4a and 4b, it includes a hose 128 for flowing gas between a hose connector 127 at one end and a face mask 129 at its other end. Face mask 129 is coupled to the mouth of patient 102 and hose connector 127 is coupled to port 121 (FIG. 1). Port 121 is a gas flow port of ventilator 105 which outputs oxygen and/or air therethrough. If ventilator 105 is a closed ventilator, then port 121 can receive gas exhaled by patient 102. Hose connector 127 is dimensioned and shaped to be received by port 121 so that it is moveable between engaged and disengaged positions relative to it. Mask 129 is held to patient 102 in a manner well-known in the art so that ventilator 105 provides ventilation for patient 102. More information about ventilators is disclosed in U.S. Pat. Nos. 6,095,138 and 4,941,469, which are incorporated herein by reference, as well as in the '005 application.

In some embodiments, repeater system 101 is carried by breathing circuit 111. In this example, it is carried by face mask 129 on its outer surface, as shown in FIG. 4b. In this way, system 101 can flow signals between resuscitation system 100 and module 103 as described above with FIG. 2b. In this example, repeater system 101 is positioned so it is up and away from the torso of patient 102 to reduce the amount of attenuation of signals S₃ and S₄. In this way, system 101 has a wider coverage area relative to patient 102 and is more likely to communicate with module 103.

In this embodiment, monitor 107 includes display 108 and is in communication with patient 102 through electrode system 113. Monitor 107 is an ElectroCardiogram (ECG) monitor which is made by many different manufacturers known in the art, such as the AED manufactures mentioned above. Monitor 107 provides many different functions, such as sensing and monitoring of the vital signs of patient 102. The vital signs generally include the heart rate and breathing rate of patient 102 and are displayed by display 108 as an ECG signal so that the user of system 100 can see them. Display 108 can also display other information, such as that provided by module 103. This information can include that corresponding to IMD 130, such as its type, model number, etc. It can also display information about patient 102, such as the medical history, contact information, past medical treatment, etc. In some examples, the vital signs are received from module 103, displayed by display 108, and flowed to communication system 106, as will be discussed in more detail below.

Electrode system 113 can be of many different types known in the art and typically includes more than two electrode pads, but only two are shown here for simplicity. For example, it can include ten electrode pads to provide a 12-lead ECG. Electrode system 113 is coupled to monitor 107 through an electrical port 123 and flows monitoring signals therethrough. In some examples, repeater system 101 is carried by electrode system 113. In this way, system 101 can flow signals between resuscitation system 100 and module 103 as described above with FIG. 2b.

In accordance with the invention, the operation of stimulator 104, ventilator 105, and/or monitor 107 is controllable in response to signal S₁ flowing between communication systems 100 and 109. It is also controllable in response to signal S₂ or signals S₃ and S₄ (FIGS. 2a-2b) flowing between systems 100 and 103. The operation can implement many different protocols, which are reprogrammable in response to these signals. In some embodiments, the protocols are programmed into system 100 and in others they are provided remotely by system 109. In this way, the protocols can be implemented by the local and remote medical personnel.

In one example of the operation of system 100, it is detected by monitor 107 that patient 102 is suffering from VF by monitoring his or her vital signs through electrode system 113. In response, communication system 106 calls remote communication system 109 with signal S₁. The medical personnel monitoring system 109 then communicate with module 103 through signals S₁ then S₂. In response, module 103 provides the medical information stored therein to remote system 109 through signals S₂ then S₁. Based on this information, the medical personnel monitoring system 109 can determine an appropriate protocol to implement using system 100. In this example, the protocol is implemented remotely in response to the medical information. In other examples, however, the medical information is provided to a user, such as the local medical personnel, assisting patient 102 locally.

In another example, the information provided by module 103 can indicate that patient 102 has an IMD, such as IMD 130. This information is provided to system 100 and communicated to the local and remote medical personnel so that they are aware of it. This information can be displayed by display 108, for example, so the local medical personnel can see it. This is useful because the presence or absence of an IMD in patient 102 often determines what protocol is most appropriate. For example, if patient 102 has a pacemaker, then it is recommended that he or she be defibrillated with an anterior-posterior electrode placement, so that the pacemaker is not damaged. The anterior-posterior electrode placement is shown in FIGS. 5 and 6 and described in more detail in the '005 application. If patient 102 does not have a pacemaker, then patient 102 can be defibrillated using an anterior-anterior (FIG. 1) or anterior posterior electrode placement.

It should be noted that system 100 can change its operation in response to changes in the condition of patient 102. For example, if system 100 determines that patient 102 is not breathing, then the remote medical personnel at system 109 can send a control signal to have system 100 provide ventilation with ventilator 105. If system 100 determines that patient 102 is suffering from VF, then the remote medical personnel can send a control signal to have system 100 provide defibrillation and/or pacing to patient 102 using stimulator 104. If system 100 determines that patient 102 needs a particular medicine, then the remote medical personnel can send a signal to module 103 through system 100 to have IMD 130, if it is an implanted infusion pump, release the desired medicine. In some situations, these steps are implemented by the local medical personnel at the scene. In either case, medical resuscitation system 100 is used to treat patient 102 in response to information provided by module 103.

As mentioned above, system 100 can be used to implement other protocols. In the United States, these protocols are typically conducted according to the most recent Advanced Cardiac Life Support guidelines for standard care, issued by the AHA. These protocols are generally updated each year and furnished in the form of algorithms.

There are currently several different protocols for cardiac patients known in the art. These include the International Advanced Cardic Life Support (ACLS) algorithm, the comprehensive (ECC) algorithm, the ventricular fibrillation/pulseless VT algorithm, pulseless electrical activity algorithm, silent heart algorithm, bradycardia algorithm, tachycardia overview algorithm, narrow-complex supraventricular tachycardia algorithm, stable ventricular tachycardia algorithm, synchronized cardioversion algorithm, among others. Although, these algorithms are recommended by the AHA, medical professionals often have their own protocols and system 100 can be programmed, to implement them using systems 100 and 109.

FIG. 5 is a sectional view of patient 102 and module 103 to illustrate how system 100 can determine the electrode pad configuration coupled to patient 102 using module 103. In this example, electrode pads 114 and 115 are in anterior apex and sternum positions, respectively, on anterior 102a of patient 102. Further, electrode pad 116 is on posterior 102b of patient 102. In this example, module 103 is implanted into patient 102 and integrated with IMD 130. Each electrode pad 114, 115, and 116 is coupled to separate connectors of stimulator 104 through electrode cables 117, 118, and 119, respectively. These separate connectors are represented by port 120 for simplicity.

The current electrode pad configuration can be determined by having module 103 output a signal so that circuitry within stimulator 104 determines which separate connector of port 120 receives the signal. In accordance with the invention, if a connector receives a signal from module 103, then its corresponding electrode pad is coupled to patient 102. For example, if signal S_{A} is received by the connector coupled to electrode cable 117, then anterior apex electrode pad 114 is coupled to patient 102. If signal S_{B} is received by the connector coupled to electrode cable 118, then anterior apex electrode pad 115 is coupled to patient 102. Further, if signal S_{C} is received by the connector coupled to electrode cable 119, then anterior apex electrode pad 116 is coupled to patient 102. It should be noted that signals S_{A}, S_{B}, and S_{C} can be the same signal or different signals that are outputted by module 103. However, they will generally be different signals when received by port 120 because they flow through different impedance paths, as discussed in more detail with FIG. 6.

If any of these signals are not received by stimulator 104, then this indicates that the corresponding electrode pad is not connected to patient 102. For example, if signals S_{A} and S_{B} are received by stimulator 104, then electrode pads 114 and 115 are coupled to patient 102 in an anterior-anterior electrode configuration. If signals S_{A} and S_{C} are received by stimulator 104, then electrode pads 114 and 116 are coupled to patient 102 in an anterior-posterior electrode configuration. If signals S_{A}, S_{B}, and S_{C} are received by stimulator 104, then electrode pads 114, 115, and 116 are coupled to patient 102.

This information is provided by system 100 to the local and remote medical personnel so that they can determine the appropriate protocol given the current electrode configuration. For example, if electrode pads 114, 115, and 116 are determined to be coupled to patient 102, then the medical personnel can choose a protocol that provides defibrillation and pacing signals between electrode pads 114 and 116 and monitoring signals between electrode pads 114 and 115. If it is determined from module 103 that patient 102 has an IMD, such as a pacemaker, then the remote medical personnel can send a message to system 100 to alert the local medical personnel that the anterior-posterior electrode configuration should be used to reduce the likelihood of damaging the pacemaker. In this example, this message is displayed by monitor 108, although it can be otherwise indicated, such as with an indicator light included with system 100.

FIG. 6 is a sectional view of patient 102 and module 103 to illustrate how system 100 can determine the transthoracic impedance of patient 102. The electrode configuration is the same as that shown in FIG. 3 for illustrative purposes. In accordance with the invention, module 103 outputs an impedance signal with a predetermined signal power and receives it with stimulator 104. In this example, the impedance signal is similar to signals S_{A}, S_{B}, and S_{C} described above. In this embodiment, stimulator 104 includes circuitry which receives the signal and determines its power and current. From this information, the circuit can determine impedances Z₁, Z₂, and Z₃ Impedances Z₁, Z₂, and Z₃ are those between module 103 and electrode pads 114, 115, and 116, respectively. Impedances Z₁, Z₂, and Z₃ are determined by the circuit using relations well-known in the art, such as Ohm's law and the relation that the signal power is proportional to the signal current multiplied by the signal voltage. Circuitry that can be used to determine the transthoracic impedance is disclosed in U.S. Pat. Nos. 6,400,984 and 4,840,177, which are incorporated herein by reference. It should be noted that these circuits can also be used to determine the electrode pad configuration as described in conjunction with FIG. 5 above.

In some examples, the defibrillation and/or pacing waveform characteristics, such as amplitude and duration, are adjusted in response to the impedance determination. This adjustment can be made by the remote and local medical personnel and it can also be made by system 100. A suitable method for adjusting the waveform characteristics in response to patient impedance is described in more detail in U.S. Pat. No. 5,999,852, which is incorporated herein by reference. This feature is useful because the transthoracic impedance is generally different for different people. Further, the transthoracic impedance for an adult is typically different from that of a child. If patient 102 is identified as a child, then lower amplitude defibrillation and pacing signals should be used to resuscitate him or her.

FIG. 7 is a side view of patient 102, showing an array of implantable medical devices included therein. In one embodiment, the array includes IMDs 130a and 130b, which are implanted into patient 102. IMDs 130a and 130b include electronic patient information modules 103a and 103b, respectively. Modules 103a and 103b are the same or similar to module 103 discussed above. Further, IMDs 130a and 130b are the same or similar to IMD 130 discussed above. In one example, IMD 130a includes an insulin infusion pump and IMD 130b includes a glucose monitoring sensor. The operation of IMDs 130a and 130b is controlled by signals flowing between modules 103a and 103b, respectively, and system 100. The signals can flow between modules 103a and 103b in a manner similar to that described in FIG. 5.

FIG. 8a is a flowchart of a method 140 of communicating with a electronic patient information module, in accordance with the present invention. Method 140 includes a step 141 of positioning an external resuscitation system near a patient carrying an electronic patient information module. A step 142 includes flowing a signal between the external resuscitation system and the electronic patient information module.

FIG. 8b is a flowchart of a method 145, in accordance with the present invention. Method 145 includes a step 146 of providing an external resuscitation system and a step 147 of resuscitating a patient in response to a signal flowed between the resuscitation system and patient module.

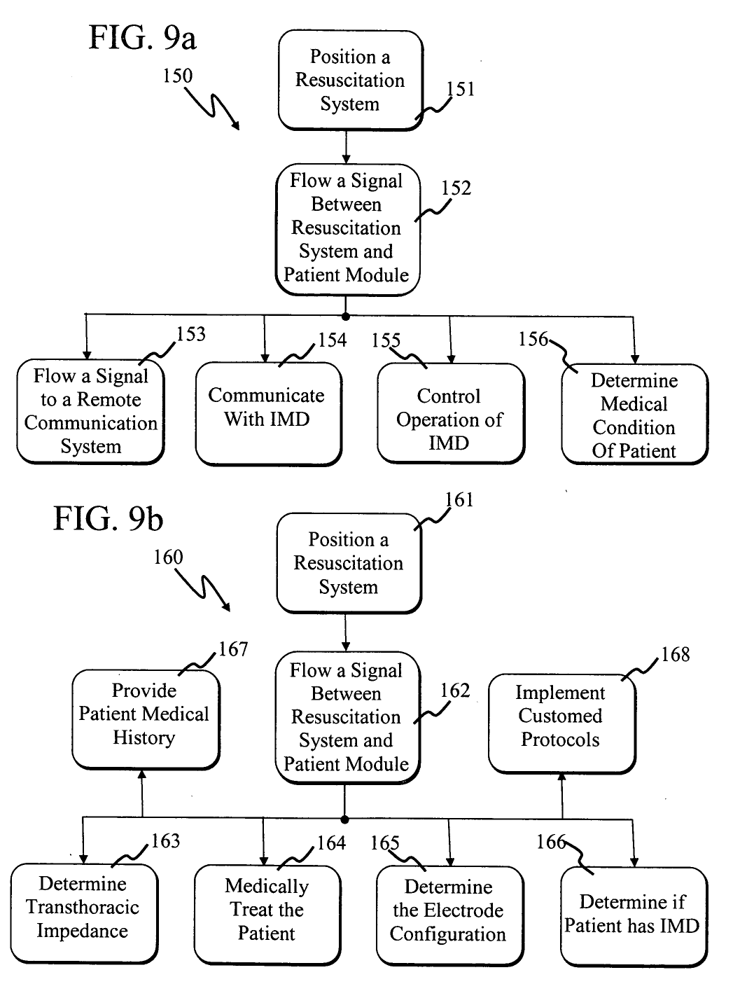

FIG. 9a is a flowchart of a method 150, in accordance with the present invention. Method 142 includes a step 151 of positioning a resuscitation system near a patient and a step 152 of flowing a signal between the resuscitation system and an electronic patient information module carried by the patient. However, after step 152, method 150 can include several other different steps in response to the signal flowing between the electronic patient information module and resuscitation system.

In some examples, method 150 includes a step 153 of flowing a signal to a remote communication system. In other examples, method 150 includes a step 154 of communicating with an IMD carried by the patient. Method 150 can also include a step 155 of controlling the operation of an IMD carried by the patient. Method 150 can further include a step 156 of determining the medical condition of the patient. It should be noted that in some embodiments, these steps can be repeated and/or control can be sent to step 152. It should also be noted that the steps and features described in conjunction with method 150 can be included in the methods described above in FIGS. 8a, 8b, and 9b.

FIG. 9b is a flowchart of a method 160, in accordance with the present invention. Method 160 includes a step 161 of positioning a resuscitation system near a patient and a step 162 of flowing a signal between the resuscitation system and an electronic patient information module carried by the patient. However, after step 162, method 160 can include several other different steps in response to the signal flowing between the electronic patient information module and resuscitation system.

In some examples, method 160 includes a step 163 of determining the transthoracic impedance of the patient using the electronic patient information module. In other examples, method 160 includes a step 164 of medically treating the patient in response to the signal. Method 160 can also include a step 165 of determining the electrode configuration of the electrodes coupled between the resuscitation system and patient using the electronic patient information module. Method 160 can also include a step 166 of determining if the patient has an IMD using the electronic patient information module.

Method 160 can also include a step 167 of providing the patient medical history which is stored in the electronic patient information module. Method 160 can further include a step 168 of implementing desired protocols in response to the signal. The desired protocols can be customized for a particular patient. It should be noted that in some embodiments, these steps can be repeated and/or control can be sent to step 162. It should also be noted that the steps and features described in conjunction with method 160 can be included in the methods described above in FIGS. 8a, 8b, and 9a.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

1. A system, comprising:
an external resuscitation system having a communication system; and
an electronic patient information module for communicating with the communication system.

2. The system of claim 1, wherein the resuscitation system provides a defibrillation signal to a patient in response to a signal from the electronic patient information module.

3. The system of claim 1, wherein the resuscitation system provides a pacing signal to a patient in response to a signal from the electronic patient information module.

4. The system of claim 1, wherein the resuscitation system provides a monitoring signal to a patient in response to a signal from the electronic patient information module.

5. The system of claim 1, further including an electrode system coupled between the external resuscitation system and a patient.

6. The system of claim 5, wherein the electronic patient information module flows a signal to the communication system through the electrode system.

7. The system of claim 5, further including a repeater system carried by the electrode system, the electronic patient information module being in communication with the communication system through the repeater system.

8. The system of claim 1, wherein the communication system flows signals between a remote communication system and the electronic patient information module.

9. The system of claim 1, wherein the external resuscitation system includes a ventilator system and a breathing circuit coupled between the ventilator system and a patient.

10. The system of claim 9, further including a repeater system carried by the breathing circuit, the electronic patient information module being in communication with the communication system through the repeater system.

11. The system of claim 1, wherein the electronic patient information module is carried externally by a patient or implanted into the patient.

12. The system of claim 1, wherein the electronic patient information module is carried by a medical device implanted in a patient.

13. A system, comprising:
a modular external resuscitation system having a communication system module and a physiological stimulator module; and
an electronic patient information module carried by a patient, the communication system module being in communication with the electronic patient information module.

14. The system of claim 13, wherein the communication system module flows a first signal to a remote communication system in response to a second signal received from the electronic patient information module.

15. The system of claim 14, wherein the modular external resuscitation system provides life support to the patient in response to the second signal.

16. The system of claim 13, further including an electrode system coupled between the patient and modular external resuscitation system, the electronic patient information module flowing the second signal to the communication system through the electrode system.

17. The system of claim 16, wherein the modular external resuscitation system provides a defibrillation signal to the patient through the electrode system in response to the first signal.

18. The system of claim 13, wherein at least two modules in the modular external resuscitation system are repeatably moveable between engaged and disengaged positions.

19. A system, comprising:
an external physiological stimulator system for resuscitating a patient;
an electronic patient information module; and
an external communication system in communication with the electronic patient information module and a remote communication system.

20. The system of claim 19, wherein the external communication system flows a first signal between the external communication system and electronic patient information module.

21. The system of claim 19, wherein the external communication system flows a second signal between the external communication system and remote communication system.

22. The system of claim 20, wherein the operation of the physiological stimulator system is controllable in response to the first signal.

23. The system of claim 21, wherein the operation of the physiological stimulator system is controllable in response to the second signal.

24. The system of claim 20, further including a ventilator system which provides ventilation to the patient in response to the first signal.

25. The system of claim 20, further including a monitor system which monitors the vital signs of the patient in response to the first signal.

26. The system of claim 20, wherein the external physiological stimulator system provides defibrillation to the patient in response to the first signal.
